(19) United States
(12) Patent Application Publication
(10) Pub. No.: US 2025/0127840 A1
(43) Pub. Date: Apr. 24, 2025

(54) METHOD FOR ACTIVATING DENDRITIC CELLS AND METHOD FOR ACTIVATING NATURAL KILLER CELLS, HELPER T CELLS, KILLER T CELLS, B CELLS, OR MACROPHAGES

(71) Applicant: TOYO SHINYAKU CO., LTD., Fukuoka (JP)

(72) Inventors: Hidetaka FUJII, Saga (JP); Takumi MORIKAWA, Saga (JP); Aiko MORITA, Saga (JP); Natsuko TANAKA, Saga (JP); Mizuki SHIRAKAWA, Saga (JP); Yuichiro ITO, Saga (JP); Kana MOTOOKA, Saga (JP); Hiroshi TOMOZAWA, Saga (JP); Shinichiro TAKASHIMA, Saga (JP)

(73) Assignee: TOYO SHINYAKU CO., LTD., Fukuoka (JP)

(21) Appl. No.: 18/893,955

(22) Filed: Sep. 23, 2024

(30) Foreign Application Priority Data
Oct. 19, 2023 (JP) 2023-180222

(51) Int. Cl. A61K 36/8998

(52) U.S. Cl. CPC A61K 36/8998

(57) ABSTRACT

It has been demanded to develop a composition for dendritic cell activation that can be easily ingested by a healthy human in daily life. The present disclosure includes a composition having a new function using young barley leaves. In a process of developing a material possessing a dendritic cell activation effect, the present inventors discovered that young barley leaves have an excellent dendritic cell activation effect, thereby completing the present disclosure.

### CROSS-REFERENCE TO RELATED APPLICATION

This application claims the priority benefit of Japan application serial no. 2023-180222, filed on Oct. 19, 2023. The entirety of the above-mentioned patent application is hereby incorporated by reference herein and made a part of this specification.

### TECHNICAL FIELD

The present disclosure relates to a composition for dendritic cell activation characterized by containing young barley leaves.

### RELATED ART

Dendritic cells (DC or DCs) are antigen-presenting cells that possess morphological characteristics of having dendritic protrusions, and are known to play important and diverse roles. There are conventional dendritic cells (cDC or cDCs) and plasmacytoid dendritic cells (pDC or pDCs). cDCs directly or indirectly activate cells such as natural killer cells (NK cells) or helper T cells, killer T cells, macrophages, and B cells, and eliminate viruses or bacteria. It is also known that killer T cells or NK cells selectively eliminate cancer cells (Patent Literature 1 and Non-Patent Literature 1), that interferon-γ secreted from activated NK cells achieves a healing effect on chronic wounds (Patent Literature 2), and that macrophages remove melanin or aged cells that have sunk into the dermis, and achieve skin whitening or anti-aging effects on skin tissue (Patent Literatures 3 and 4). Hence, by activating cDCs, effects such as maintenance of immune function, antiviral action, anticancer action, promotion of healing of chronic wounds, skin whitening action, and anti-aging action on skin are exerted. Furthermore, it is known that dendritic cells are essential for embryo implantation and their absence leads to infertility (Non-Patent Literature 2). Therefore, it is also expected that cDC activation may be useful for infertility treatment.

On the other hand, pDCs demonstrate a high ability of producing interferon-α being an important cytokine for antiviral defense, and possess a function of activating cells such as NK cells or helper T cells, killer T cells, macrophages, and B cells. Hence, like cDCs, by activating pDCs, effects such as maintenance of immune function, antiviral action, anticancer action, promotion of healing of chronic wounds, skin whitening action, and anti-aging action on skin are exerted.

In this way, if dendritic cells can be activated, various effects are exerted. Therefore, it has been demanded to develop a method for activating dendritic cells. In recent years, a “dendritic cell vaccine therapy” has been developed which involves culturing dendritic cells ex vivo, expanding them, and then returning them to the patient's body (Non-Patent Literature 3). However, since the “dendritic cell vaccine therapy” is aimed at cancer treatment, it cannot be performed by a healthy human for purposes such as immune function maintenance, skin whitening, or skin aging prevention in daily life. Hence, it has been demanded to develop a composition for dendritic cell activation that can be easily ingested by a healthy human in daily life.

### PRIOR-ART LITERATURES

### Patent Literatures

[Patent Literature 1] Japanese Patent No. 5742050
[Patent Literature 2] Japanese Patent No. 4616291
[Patent Literature 3] Japanese Patent Laid-Open No. 2005-281205
[Patent Literature 4] Japanese Patent Laid-Open No. 2021-195305

### Non-Patent Literatures

[Non-Patent Literature 1] Website of Shinyokohama Kato Clinic, Medical Corporation Shinjukai, “Structure of Activated Lymphocytes (Killer T Cells)”, URL: https://katoclinic.info/clinic-treatment/lak/system/, accessed on Oct. 6, 2023
[Non-Patent Literature 2] The Journal of Clinical Investigation, 2008 December; 118 (12): 3954-65
[Non-Patent Literature 3] Website of Kyushu Koseikai Clinic, Dendritic Cell Vaccine Therapy, URL: https://kyushukouseikai.or.jp/dc/, accessed on Oct. 6, 2023

### SUMMARY OF THE INVENTION

### Problems to be Solved by the Invention

The present disclosure is made in view of the above problems, and a problem to be solved by the present disclosure is to provide a composition including a new function using young barley leaves.

### Means for Solving the Problems

In a process of developing a material possessing a dendritic cell activation effect, the present inventors discovered that young barley leaves have an excellent dendritic cell activation effect, thereby completing the present disclosure.

That is, the present disclosure is as follows.

[1]

A composition for dendritic cell activation, characterized by containing young barley leaves.

[2]

The composition for dendritic cell activation, characterized in that young barley leaf-derived dietary fiber is an active ingredient.

[3]

The composition for dendritic cell activation as described in [1] or [2], characterized by being used in maintaining immune function.

[4]

The composition for dendritic cell activation as described in [1] or [2], characterized in that the composition is foods with function claims.

[5]

The composition for dendritic cell activation as described in [1] or [2], characterized in that the dendritic cell is a conventional dendritic cell (CDC).

[6]

The composition for dendritic cell activation as described in [1] or [2], characterized in that the young barley leaves are young barley leaf powder.

[7]

Foods with function claims, which contains young barley leaf powder, in which the foods with function claims displays an indication that young barley leaf-derived dietary fiber acts on conventional dendritic cell (CDC) and is useful for maintenance of a healthy human immune function.

[8]

Foods with function claims, which contains young barley leaf powder, in which the foods with function claims displays an indication that young barley leaf-derived dietary fiber acts on conventional dendritic cell (CDC).

[9]

The foods with function claims as described in [8], characterized by being used to facilitate maintenance of a healthy human immune function.

[10]

The composition for dendritic cell activation as described in [1], characterized in that a daily ingestion amount of the young barley leaves is 0.25 g or more.

[11]

The composition for dendritic cell activation as described in [2], characterized in that a daily ingestion amount of young barley leaf-derived dietary fiber is 0.125 g or more.

[12]

Use of young barley leaves for producing a composition for dendritic cell activation.

[13]

A method for activating dendritic cells in vivo in an individual, including a step of causing the individual to orally ingest young barley leaves.

[14]

A composition for natural killer cell activation, a composition for helper T cell activation, a composition for killer T cell activation, a composition for B cell activation, or a composition for macrophage activation, characterized by containing young barley leaves as an active ingredient.

[15]

Use of young barley leaves for producing a composition for natural killer cell activation, a composition for helper T cell activation, a composition for killer T cell activation, a composition for B cell activation, or a composition for macrophage activation.

[16]

A method for activating natural killer cells, helper T cells, killer T cells, B cells, or macrophages in vivo in an individual, including a step of causing the individual to orally ingest young barley leaves.

[17]

A composition for interleukin-12 secretion promotion or a composition for interferon-γ secretion promotion, characterized by containing young barley leaves as an active ingredient.

[18]

Use of young barley leaves for producing a composition for interleukin-12 secretion promotion or a composition for interferon-γ secretion promotion.

[19]

A method for promoting secretion of interleukin-12 or interferon-γ in vivo in an individual, including a step of causing the individual to orally ingest young barley leaves.

[20]

A method for activating dendritic cells by causing a human to ingest a composition containing young barley leaves.

[21]

The method as described in [20], in which immune function is maintained by activating dendritic cells.

[22]

The method for activating dendritic cells by causing a human to ingest a composition containing young barley leaf-derived dietary fiber.

[23]

The method as described in [22], in which immune function is maintained by activating dendritic cells.

[24]

A method for activating natural killer cells, helper T cells, killer T cells, B cells, or macrophages by causing a human to ingest young barley leaves.

### Effects of the Invention

The composition of the present disclosure is capable of promoting the secretion of a cytokine such as interleukin-12 or interferon-γ by activating dendritic cells, and is capable of activating natural killer cells, helper T cells, killer T cells, B cells, and macrophages. Accordingly, immune function can be maintained. Furthermore, effects such as antiviral action, anticancer action, promotion of healing of chronic wounds, skin whitening action, and anti-aging action on skin can be exerted.

### DESCRIPTION OF THE EMBODIMENTS

### 1. Young Barley Leaves

The present disclosure is characterized by containing young barley leaves. Thought to originate from Central Asia, barley (Hordeum vulgare L.) is a poaceous annual or biennial plant, and is broadly classified into two-rowed barley, six-rowed barley or the like based on the shape of its ears. Barley as a grain is often used in the form of its seed; however, the present disclosure uses young leaves of barley. Comparing these two, a barley seed is mainly composed of starch (carbohydrates), whereas a young barley leaf is mainly composed of dietary fiber and protein, and their component compositions are totally different from each other. A barley variety used for the young barley leaves of the present disclosure is not particularly limited as long as it is commonly available, and young barley leaves of any variety of two-rowed barley, six-rowed barley or the like may be used. Young barley leaves refer to leaves of barley before start of ear emergence. In the present disclosure, stems may be included along with young leaves.

As the young barley leaves, for example, a ground product, a juice, an extract, or an extraction residue, may be used. Examples of the ground product include dried powder (young barley leaf powder), a finely chopped product, and a dried product (dried finely chopped product) thereof. The juice or the extract may be in a liquid form, and juice powder is preferable. The juice powder of young barley leaves may be obtained by, for example, concentrating juice of young leaves at a low temperature to concentrate the solid content, and freeze-drying or spray-drying the concentrate liquid. As the extract, for example, water (warm water, hot water), ethanol, or aqueous ethanol, may be used. As the extraction residue, for example, a precipitate obtained by suspending young barley leaf powder in water, stirring and shaking the suspension, and then performing centrifugation, may be used. The extraction residue is a fraction that is removed in a process of obtaining the juice or the extract, and is able to exhibit effects of the present disclosure due to its rich content of young barley leaf-derived dietary fiber. From the viewpoint of being able to reliably exhibit effects of the present disclosure by containing young barley leaf-derived dietary fiber and being able to be produced at low cost, the young barley leaves used in the present disclosure is preferably a ground product. Among the ground products, dried powder (young barley leaf powder) is particularly preferable.

A young barley leaf fermented product obtained by fermentation of young barley leaves by microorganisms such as lactic acid bacteria is not included in the young barley leaves of the present disclosure. In the case of fermentation, a protein or sugar or the like is broken down by the microorganisms, and its component undergoes a chemical change. Since the young barley leaves after fermentation change into a different substance having a component composition totally different from that before fermentation, and lack equivalence compared to the young barley leaves before fermentation, the young barley leaves after fermentation do not correspond to the young barley leaves of the present disclosure.

In the case of using young barley leaf powder, for example, young barley leaves dried to have a moisture content of 10 mass % or less, preferably 5 mass % or less, may be used.

The young barley leaf powder used in the present disclosure preferably has a median diameter of 5 μm or more and 50 μm or less, and more preferably 8 μm or more and 30 μm or less. The median diameter refers to a particle size at a cumulative 50% point in a particle size distribution of the powder, and specifically, a particle size at a cumulative 50% (×50) point in a particle size distribution measured using LMS-300 or LMS-350 manufactured by Seishin Enterprise Co., Ltd. that is a laser diffraction and scattering particle size distribution analyzer.

From the viewpoint of being able to reliably exhibit effects of the present disclosure, the young barley leaves used in the present disclosure preferably contain dietary fiber in an amount of 10 mass % or more, more preferably 20 mass % or more, and even more preferably 30 mass % or more. The upper limit may be, for example, 80 mass %. The dietary fiber referred to herein is a total amount of insoluble dietary fiber and water-soluble dietary fiber. The amount of dietary fiber is measured by the Prosky method.

From the viewpoint of being able to reliably exhibit effects of the present disclosure, the young barley leaves used in the present disclosure preferably contain insoluble dietary fiber in an amount of 5 mass % or more, more preferably 10 mass % or more, and even more preferably 20 mass % or more. The upper limit may be, for example, 70 mass %. The amount of insoluble dietary fiber is measured by the modified Prosky method.

To achieve the amount of dietary fiber or the amount of insoluble dietary fiber within the above range, in a method for producing young barley leaves, young barley leaf powder may be prepared by pulverizing the young barley leaves without performing juice extraction or extraction.

### 2. Dendritic Cells

In the present disclosure, dendritic cells refer to a concept including conventional dendritic cells (cDC or cDCs) and plasmacytoid dendritic cells (pDC or pDCs). In the present disclosure, activation of dendritic cells means that some function possessed by dendritic cells is enhanced, or the amount of genes or proteins expressed in dendritic cells is amplified. Specific examples include promotion of dendritic cell maturation, enhancement of antigen uptake ability, enhancement of antigen presentation ability, T cell activation ability, and enhancement of production of cytokines such as interleukin-12. The composition for dendritic cell activation of the present disclosure is particularly excellent in activating conventional dendritic cells (cDCs).

When cDCs are activated, expression of MHC [abbreviation of major histocompatibility complex; called human leukocyte antigen (HLA) in humans] is enhanced, and production of interleukin-12 (IL-12) is further enhanced. IL-12 is a protein having a molecular weight of about 70,000, and is responsible for differentiation induction (T cell activation) from naive T cells to helper T cells, or activation of NK cells. The helper T cells activated by IL-12 produce interferon gamma (IFN-γ).

IFN-γ is a dimeric protein, and serves to transmit a command from helper T cells to killer T cells or B cells. By receiving this command, the killer T cells or B cells can be activated and can attack pathogens such as viruses or bacteria. Furthermore, IFN-γ activates macrophages, promoting the macrophages to ingest, digest, and sterilize bacteria that have invaded the body or melanin or aged cells or the like that have sunk into the dermis. In this way, when cDCs are activated, the secretion of interleukin-12 is promoted, helper T cells are activated, the secretion of interferon-γ from the helper T cells is promoted, and natural killer cells or killer T cells, B cells, and macrophages are activated.

On the other hand, pDCs produce interferon alpha (IFN-α) being an important cytokine for antiviral defense. Since IFN-α is a signal substance in which various immune cells work, the production of IFN-α activates natural killer cells or helper T cells, killer T cells, B cells, and macrophages.

Through the aforementioned mechanism, by activation of dendritic cells, natural killer cells, helper T cells, killer T cells, B cells, and macrophages are activated, and effects such as maintenance of immune function, antiviral action, anticancer action, promotion of healing of chronic wounds, skin whitening action, and anti-aging action on the skin are achieved.

### 3. Composition of the Present Disclosure

### <Composition for Dendritic Cell Activation>

The composition for dendritic cell activation of the present disclosure contains young barley leaves and is used for activation of dendritic cells. In these aspects, the composition for dendritic cell activation is not particularly limited as long as it, as a product, can be distinguished from other products. For example, any of a body, packaging, a manual, and a promotional material (advertising medium) of a product according to the present disclosure, which displays the function of activating dendritic cells, is included in the scope of the present disclosure. The composition for dendritic cell activation of the present disclosure may display young barley leaves or an ingredient (such as young barley leaf-derived dietary fiber) derived from young barley leaves as an active ingredient, but is not limited to displaying young barley leaves or an ingredient (such as young barley leaf-derived dietary fiber) derived from young barley leaves as an active ingredient on the packaging or the like of the product. For example, the composition for dendritic cell activation may not specify an active ingredient. It is sufficient that a composition for dendritic cell activation having young barley leaf-derived dietary fiber as an active ingredient contains young barley leaf-derived dietary fiber, and it is not necessary to use fractionated young barley leaf-derived dietary fiber. For example, by blending of young barley leaves containing young barley leaf-derived dietary fiber, such as young barley leaf powder (ground powder), the composition for dendritic cell activation having young barley leaf-derived dietary fiber as an active ingredient can be obtained. General foods that are manufactured and sold with a suggested use are included in the scope of the present disclosure. For example, foods sold with testimonials put on a website or the like mentioning dendritic cell activation, as personal impressions of those who have ingested the foods, are also included in the scope of the present disclosure. Foods with nutrient function claims that use papers demonstrating dendritic cell activation by young barley leaves as scientific evidence for functionality and have young barley leaves or an ingredient derived from young barley leaves as a functional ingredient, and that claim functionality related to the maintenance of immune function, are also included in the scope of the present disclosure. In the present application, the maintenance of immunity is a concept including suppressing deterioration of immune function and/or improving immune function.

Specifically, in so-called health foods, examples of the claims include: “activate dendritic cells”, “activate cDCs”, “activate pDCs”, “help activate dendritic cells”, “help activate cDCs”, “help activate pDCs”, “serve to activate dendritic cells”, “serve to activate cDCs”, “serve to activate pDCs”, “make dendritic cells active”, “make cDCs active”, “make pDCs active”, “help make dendritic cells active”, “help make cDCs active”, “help make pDCs active”, “serve to make dendritic cells active”, “serve to make cDCs active”, “serve to make pDCs active”, “act on dendritic cells”, “act on cDCs”, “act on pDCs”, “aid the function of dendritic cells”, “aid the function of cDCs”, “aid the function of pDCs”, “act on dendritic cells and serve to maintain a healthy human immune function”, “act on conventional dendritic cells (cDCs) and serve to maintain a healthy human immune function”, “act on plasmacytoid dendritic cells (pDCs) and serve to maintain a healthy human immune function”, “aid the function of dendritic cells and serve to maintain a healthy human immune function”, “serve to maintain a healthy human immune function by acting on conventional dendritic cells (cDCs)”, “serve to maintain a healthy human immune function by acting on plasmacytoid dendritic cells (pDCs)”, “serve to maintain a healthy human immune function by acting on the function of dendritic cells”, “aid the function of conventional dendritic cells (cDCs) and serve to maintain a healthy human immune function”, “aid the function of plasmacytoid dendritic cells (pDCs) and serve to maintain a healthy human immune function”, “act on dendritic cells and help maintain a healthy human immune function”, “act on conventional dendritic cells (cDCs) and help maintain a healthy human immune function”, “act on plasmacytoid dendritic cells (pDCs) and help maintain a healthy human immune function”, “act on dendritic cells and serve to maintain immune function”, “act on conventional dendritic cells (cDCs) and serve to maintain immune function”, “act on plasmacytoid dendritic cells (pDCs) and serve to maintain immune function”, “aid the function of dendritic cells and serve to maintain immune function”, “serve to maintain immune function by acting on conventional dendritic cells (cDCs)”, “serve to maintain immune function by acting on plasmacytoid dendritic cells (pDCs)”, “serve to maintain immune function by acting on the function of dendritic cells”, “aid the function of conventional dendritic cells (cDCs) and serve to maintain immune function”, “aid the function of plasmacytoid dendritic cells (pDCs) and serve to maintain immune function”, “act on dendritic cells and help maintain immune function”, “act on conventional dendritic cells (cDCs) and help maintain immune function”, and “act on plasmacytoid dendritic cells (pDCs) and help maintain immune function”.

As described above, the composition for dendritic cell activation of the present disclosure promotes the secretion of interleukin-12 or the like, or activates natural killer cells or the like, thereby achieving the effects such as maintenance of immune function, antiviral action, anticancer action, promotion of healing of chronic wounds, skin whitening action, and anti-aging action on the skin. Hence, the composition for dendritic cell activation of the present disclosure can be used for the purposes of maintaining immune function, acting against viruses, acting against cancers, promoting healing of chronic wounds, whitening skin, and acting against aging of the skin.

According to the composition of the present disclosure, by activation of dendritic cells, the secretion of interleukin-12 is promoted or the secretion of interferon-γ from activated helper T cells is promoted. Hence, the composition of the present disclosure can be used as a composition for promoting secretion of interleukin-12 or a composition for promoting secretion of interferon-γ. According to the composition of the present disclosure, by activation of dendritic cells, natural killer cells, helper T cells, killer T cells, B cells, and macrophages are activated. Hence, the composition of the present disclosure can be used as a composition for natural killer cell activation, a composition for helper T cell activation, a composition for killer T cell activation, a composition for B cell activation, or a composition for macrophage activation.

### <Dosage Form or the Like of Composition>

The composition of the present disclosure may be used as an oral composition. The form of the oral composition is not particularly limited, and examples thereof include a food and beverage product, a quasi-drug, and a pharmaceutical. From the viewpoint of daily ingestion of young barley leaves to supplement daily nutrition, the composition of the present disclosure is preferably used as a food and beverage product. The form of a food and beverage product composition is not particularly limited, and examples thereof include foods with function claims, a food for specified health uses, a health food, and a general food and beverage product. From the viewpoint of making it easy for consumers to understand effects of the present disclosure, the foods with function claims and the food for specified health uses are preferable, and the foods with function claims is most preferable.

Examples of the form of the composition of the present disclosure include a powder form, a granule form, a liquid form, a tablet form, and a capsule form. The composition of the present disclosure may also be in the form of jelly or cookie or the like. As described above, the young barley leaves of the present disclosure are not a fermented product and have a good taste. Thus, the young barley leaves of the present disclosure are suitable for ingestion in a form in which the taste is perceived. Hence, the powder form, the granule form, the liquid form, and the tablet form are preferable; the powder form, the granule form, and the liquid form are particularly preferable.

The content of young barley leaves in the composition of the present disclosure may be appropriately configured in a range that achieves effects of the young barley leaves. For example, the content of young barley leaves in the composition of the present disclosure on a dry mass basis may be 0.01 to 100 mass %, preferably 0.1 to 100 mass %, and more preferably 1 to 100 mass %.

The content of young barley leaf-derived dietary fiber in the composition of the present disclosure may be appropriately configured in a range that achieves effects of the young barley leaf-derived dietary fiber. For example, the content of young barley leaf-derived dietary fiber in the composition of the present disclosure on a dry mass basis may be 0.01 to 80 mass %, preferably 0.1 to 75 mass %, and more preferably 1 to 70 mass %.

An ingestion amount of young barley leaves in the present disclosure is not particularly limited. From the viewpoint of being able to reliably exhibit effects of the present disclosure, the ingestion amount of young barley leaves per day for an adult is preferably 0.25 g or more, more preferably 0.5 g or more, and particularly preferably 1.0 g or more. The upper limit is, for example, 30 g, preferably 20 g, and more preferably 10 g.

An ingestion amount of young barley leaf-derived dietary fiber in the present disclosure is not particularly limited. From the viewpoint of being able to reliably exhibit effects of the present disclosure, the ingestion amount of young barley leaf-derived dietary fiber per day for an adult is preferably 0.125 g or more, more preferably 0.25 g or more, and particularly preferably 0.5 g or more. The upper limit is, for example, 15 g, preferably 10 g, and more preferably 5 g.

The composition of the present disclosure may be appropriately designed such that a daily ingestion amount of young barley leaves or young barley leaf-derived dietary fiber is the above ingestion amount. The composition of the present disclosure may be ingested in a form in which the above ingestion amount is consumed in one dose, or in a form in which the above ingestion amount is consumed separately in multiple doses. That is, for example, as a daily portion, the composition of the present disclosure may be contained in one container or separately in multiple (2 to 4) containers.

The composition of the present disclosure may be added with ingredients other than young barley leaves as required, and be produced according to a known method.

### EXAMPLES

The present disclosure will be described in more detail below by examples. However, the present disclosure is not limited to these examples and can take various forms as long as the problems to be solved by the present disclosure can be solved.

### Test 1. Evaluation of Dendritic Cell Activation by Young Barley Leaves

To evaluate the dendritic cell activation effect of young barley leaves, a gene dosage of cytokines secreted from dendritic cells was evaluated using dendritic cells differentiated from mouse-derived bone marrow cells by a test described below.

### Test 1 (A). Evaluation of TNF-α Gene Expression

As a cytokine, tumor necrosis factor-α (TNF-α) was used as an indicator. TNF-α is one of the powerful mediators of biological responses that is rapidly secreted from cDCs and pDCs during foreign invasion, and serves as an indicator of dendritic cell activation.

### <Test Substance>

### (Young Barley Leaf Powder)

As a raw material, the above-ground parts (leaves and stems) of barley harvested at a height of about 30 cm were used. After washing to remove adhered soil and cutting into pieces of about 5 cm in size, the resultant was blanched only once in hot water at 95° C. for about 100 seconds, then cooled with cold water. The obtained young barley leaves were dried with warm air in a dryer and coarsely ground to about 1 mm in size. Furthermore, the resultant was finely ground using a jet mill grinder to produce young barley leaf powder (having a median diameter of about 20 μm, a moisture content of 5 mass % or less, and a young barley leaf-derived dietary fiber content of 40 mass %).

### (Test Substance Other Than Young Barley Leaf Powder)

As test substances other than young barley leaf powder, the following were used.

Young barley leaf juice powder: powder obtained by mixing the juice of young barley leaves with dextrin and drying was used (having a young barley leaf-derived dietary fiber content of 2 mass %).
Cellulose: commercially available cellulose was used.
Vitamin D3: Vitamin D3 commercially available as a reagent was used.
β-glucan (derived from barley seeds): β-glucan (purified product derived from barley seeds; having a purity of 98%) commercially available as a reagent was used.

### <Test Method>

After dissection of a C57BL/6J mouse (8 weeks old), bone marrow mononuclear cells were sampled from the femur and tibia, and cultured using a differentiation induction medium (RPMI 1640 medium containing 10% fetal bovine serum, 100 μM of 2-mercaptoethanol, and 100 ng/ml of Flt3 ligand). Specifically, the cells were seeded in a 96-well plate at 100 μL/well so that 3×10⁵ cells/well was achieved, and cultured for 7 days in a 5% CO2 incubator (“CO2 Incubator” manufactured by ASTEC) at 37° C., with the medium changed every 3 to 4 days, thereby differentiating the cells into bone marrow-derived dendritic cells (including both cDCs and pDCs).

After the medium was removed from each well, 100 μL of a differentiation induction medium containing 0.5% DMSO, which was prepared so that a total concentration of the test substances was 10 μg/mL (for young barley leaf juice powder, the concentration of young barley leaf juice ingredient excluding dextrin was 10 μg/mL), was added to each well and cultured for 72 hours. The ratios of each test substance are shown in Table 1.

After that, the medium was removed from each well, and RNA was collected using the RNeasy Mini Kit (manufactured by QIAGEN). From the obtained RNA, a gene expression level (mRNA expression level) of TNF-α was measured by the real-time PCR method using the One Step TB Green® PrimeScript™ RT-PCR Kit II <Perfect Real Time> (manufactured by TaKaRa). For a primer, Mm_Il12B_1_SG QuantiTect Primer Assay and Mm_Tnf1_SG QuantiTect Primer Assay (manufactured by QIAGEN) were used.

### <Test Result>

The TNF-α gene expression level (relative value) of the examples and comparative examples was evaluated, with the value (TNF-α/β-actin) of a control with no addition of test substance set to 1. The result is shown in Table 1. In Examples 1 and 2 containing young barley leaves, the TNF-α gene expression level increased compared to the control. Since TNF-α serves as an indicator of dendritic cell activation, it is suggested that young barley leaves activate dendritic cells.

In particular, in Example 1 containing young barley leaf powder, a relatively pronounced promotion of TNF-α gene expression was observed. Dietary fiber (cellulose) and Vitamin D, which are known to have immune-stimulating effects, did not promote TNF-α gene expression (Comparative Examples 1 and 2). From this, it is confirmed that an ingredient having immune-stimulating effects did not necessarily activate dendritic cells.

TABLE 1

| | | | | | Comparative | Comparative | Comparative |
|---|---|---|---|---|---|---|---|
| | | Control | Example 1 | Example 2 | Example 1 | Example 2 | Example 3 |
| Ratio | Young barley leaf powder | | 100 | | | | |
| (%) | Young barley leaf juice | | | 100 | | | |
| of | powder (as juice ingredient) | | | | | | |
| each | Cellulose | | | | 100 | | |
| test | Vitamin D3 | | | | | 100 | |
| substance | β-glucan | | | | | | 100 |
| | Total | 0 | 100 | 100 | 100 | 100 | 100 |

| | | | | | | |
|---|---|---|---|---|---|---|
| TNF-α gene expression level | 1.00 | 1.40 | 1.29 | 1.05 | 0.96 | 1.17 |
| (relative value) | | | | | | |

### Test 1 (B). Evaluation of IL-12 Gene Expression

As a cytokine, interleukin-12 (IL-12) was used as an indicator. As described above, IL-12 is a cytokine secreted by conventional dendritic cells (cDCs) and serves as an indicator of dendritic cell activation.

### <Test Substance>

The same young barley leaf powder as in Test 1 (A) was used.

### <Test Method>

Bone marrow mononuclear cells were collected from the femur and tibia of a 8-week-old C57BL/6J mouse, and cultured in a differentiation induction medium (RPMI 1640 medium containing 10% fetal bovine serum, 100 UM of 2-mercaptoethanol, 100 ng/ml of Flt3 ligand, 100 units/mL of penicillin, and 100 μg/mL of streptomycin), and bone marrow-derived dendritic cells were induced. After 7 days of culture, a differentiation induction medium containing young barley leaf powder at a final concentration of 10 μg/ml or a differentiation induction medium containing no young barley leaf powder as a control was added. After 72 hours of culture, RNA was collected using the RNeasy Mini Kit (QIAGEN), real-time PCR was performed using the One

Step TB Green® PrimeScript RT-PCR Kit II (Perfect Real Time) (TaKaRa), and an mRNA expression level of IL-12 was evaluated. The mRNA expression level of β-actin was used as an endogenous control. For a primer, Mm_Il12B_1_SG QuantiTect Primer Assay and Mm Actb_1_SG QuantiTect Primer Assay (QIAGEN) were used.

### <Test Result>

The IL-12 gene expression level (relative value) was evaluated, with the value (IL-12/β-actin) of the control with no addition of test substance set to 1. The result is shown in Table 2. Compared to the control, the group with addition of young barley leaf powder demonstrated an increase in the IL-12 gene expression level, and it can be confirmed that the dendritic cells were activated.

TABLE 2

| | | Young barley |
|---|---|---|
| | Control | leaf powder |
| IL-12 gene expression | 1.0 | 4.1 |
| level (relative value) | | |

### Test 2. Evaluation of Dendritic Cell Activation by Young Barley Leaf-Derived Dietary Fiber

The result shown in Test 1 (A) indicated that young barley leaf powder (ground powder of young barley leaves) had a higher dendritic cell activation effect than young barley leaf juice powder obtained by concentrating a water-soluble ingredient of young barley leaves. It is inferred from this result that young barley leaf-derived dietary fiber removed in a juicing process of young barley leaves may contribute to dendritic cell activation. To confirm this, the dendritic cell activation effect of young barley leaf-derived dietary fiber was evaluated by the following test.

### Test 2 (A). Evaluation of Il-12 Gene Expression Level in Dendritic Cells

To evaluate the dendritic cell activation effect of young barley leaf-derived dietary fiber, a gene dosage of cytokines secreted from dendritic cells was evaluated by a test described below. As a cytokine, interleukin-12 (IL-12) was used as an indicator.

### <Test Substance>

Young barley leaf-derived dietary fiber was fractionated from young barley leaf powder obtained by the method described in Test 1 and used as a test substance.

### <Test Method>

Bone marrow mononuclear cells were collected from the femur and tibia of a C57BL/6J mouse (7 weeks old), and cultured in a differentiation induction medium (RPMI 1640 medium containing 10% fetal bovine serum, 55 μM of 2-mercaptoethanol, 20 ng/ml of GM-CSF, 1% penicillin, and 10 mM of HEPES), and bone marrow-derived dendritic cells were induced. After 6 days of culture in a 5% CO2 incubator at 37° C., a medium (RPMI 1640 medium containing 10% fetal bovine serum, 55 μM of 2-mercaptoethanol, 1% penicillin, and 10 mM of HEPES) for test substance addition containing young barley leaf-derived dietary fiber, a medium (control) for test substance addition containing no young barley leaf-derived dietary fiber, and a medium (positive control) for test substance addition containing zymosan, were prepared. The concentrations of each test substance are as shown in Table 3. After 5 hours of culture, RNA was collected using the RNeasy Mini Kit (QIAGEN), real-time PCR was performed using the One Step TB Green® PrimeScript RT-PCR Kit II (Perfect Real Time) (TaKaRa), and a gene expression level (mRNA expression level) of IL-12 was evaluated. The mRNA expression level of Rn18s was used as an endogenous control. For a primer, Mm_1112B_1_SG QuantiTect Primer Assay and Mm Rn18S_3_SG QuantiTect Primer Assay (QIAGEN) were used.

### <Test Result>

The IL-12 gene expression level (relative value) of the examples and contrast example was evaluated, with the value (IL-12/Rn18s) of the control with no addition of test substance set to 1. The result is shown in Table 3. With the young barley leaf-derived dietary fiber, the IL-12 gene expression level increased, and the expression level was significantly higher compared to zymosan being a known dendritic cell activator. It is suggested from this result that young barley leaf-derived dietary fiber contributes as an active ingredient of young barley leaves to dendritic cell activation.

TABLE 3

| | | | Contrast | | | |
|---|---|---|---|---|---|---|
| | | Control | Example | Example 3 | Example 4 | Example 5 |
| Concentration | Zymosan | | 10 | | | |
| (μg/ml) of | (positive control) | | | | | |
| test substance | Young barley leaf-derived | | | 2 | 5 | 10 |
| | dietary fiber | | | | | |
| | Total | 0 | 10 | 2 | 5 | 10 |

| | | | | | |
|---|---|---|---|---|---|
| IL-12 gene expression level (relative value) | 1.0 | 1.9 | 12.9 | 32.7 | 56.8 |

### Test 2 (B). Evaluation of Amount of Il-12 Produced in Dendritic Cells

To confirm a correlation between gene and protein expression levels of IL-12, the amount of IL-12 (as protein) secreted from dendritic cells was evaluated by a test described below using the young barley leaf-derived dietary fiber prepared in Test 2 (A).

### <Test Method>

The dendritic cells were differentiated using bone marrow mononuclear cells sampled from a C57BL/6J mouse (8 weeks old) in the same manner as in Test 2 (A). After 6 days of culture in a 5% CO2 incubator at 37° C., a medium for test substance addition containing young barley leaf-derived dietary fiber and a medium (control) for test substance addition containing no young barley leaf-derived dietary fiber were prepared to achieve the final concentrations shown in Table

4. After 48 hours of culture, the culture supernatant was collected, and the amount of IL-12 was evaluated using the Mouse IL-12p40 Simple Step ELISA Kit (manufactured by abcam).

### <Test Result>

The measured IL-12 concentrations are shown in Table 4. In a comparison of the IL-12 concentrations, while the control had an IL-12 concentration of 17.2 pg/mL, Examples 6 to 8 had IL-12 concentrations of 75.7 pg/mL (4.4 times that of the control), 140.7 pg/mL (8.2 times that of the control), and 255.2 pg/mL (14.8 times that of the control), respectively. It is suggested from this result that a correlation is present between the gene and protein expression levels of IL-12, and that the amount of IL-12 produced increases as a result of activation of dendritic cells by young barley leaf-derived dietary fiber.

TABLE 4

| | | Control | Example 6 | Example 7 | Example 8 |
|---|---|---|---|---|---|
| Concentration (μg/ml) of | Young barley leaf-derived dietary fiber | | 2 | 5 | 10 |
| test substance | Total | 0 | 2 | 5 | 10 |

| | | | | |
|---|---|---|---|---|
| Concentration (pg/mL) of IL-12 p40 | 17.2 | 75.7 | 140.7 | 255.2 |

### Test 3. Evaluation of Natural Killer Cell Activation by Young Barley Leaves

It is conceivable that young barley leaves activate natural killer cells (NK cells) by activating dendritic cells and promoting the secretion of IL-12. On the other hand, it was unclear whether young barley leaves also directly act on NK cells. To confirm this, cytotoxicity (activity by which NK cells kill target cells) of NK cells was evaluated by a test described below.

### <Test Substance>

For Spirulina, ground powder of Spirulina was used. For the other test substances, the same ones as in Test 1 (A) were used.

### <Test Method>

After dissection of a C57BL/6J mouse (8 weeks old), spleen cells were isolated, and a cell suspension prepared by dilution in a 10% fetal bovine serum-RPMI 1640 medium was seeded at 100 L/well so that the cells were seeded at 5×10⁶ cells/well in a 96-well plate. To the seeded cells, a medium (0.5% DMSO-10% fetal bovine serum-RPMI 1640 medium) prepared to have a total concentration of test substances of 1 μg/mL (for young barley leaf juice powder, the concentration of young barley leaf juice ingredient excluding dextrin was 1 μg/mL) was added at 100 μL/well, and the resultant was cultured for 18 hours in a 5% CO2 incubator (“CO2 Incubator” manufactured by ASTEC) at 37° C. The ratios of each test substance are shown in Table 5.

After culture, the plate was centrifuged (at 1,000 rpm for 10 minutes) using a centrifuge. 150 μL of the supernatant was removed, and 150 μL of 1% BSA-RPMI 1640 was added. This process was repeated once more. After that, YAC-1 cells (stained with calcein) sensitive to NK cells were added as target cells, and the resultant was cultured for 4 hours.

After culture, the plate was centrifuged (at 1,000 rpm for 10 minutes) using a centrifuge, and the supernatant was collected in a 96-well black plate. The collected supernatant was measured for fluorescence intensity (at an excitation wavelength of 490 nm and a fluorescence wavelength of 515 nm) using a microplate reader (“SpectraMax i3x” manufactured by Molecular Devices), and the NK cell cytotoxicity was evaluated.

### <Test Result>

The NK cell cytotoxicity (relative value) is shown in Table 5 with the value of the control set to 1. A higher relative value of NK cell cytotoxicity indicates greater activation of NK cells. In Examples 9 and 10 containing young barley leaves, the NK cell cytotoxicity was higher compared to the control. From this, it is suggested that young barley leaves not only promote the secretion of IL-12 through dendritic cell activation and activates NK cells, but also directly act on the NK cells and activate the NK cells. Even if dietary fiber (cellulose), Vitamin D, or Spirulina, which are known to have immune-stimulating effects, was added, the NK cells were not activated.

TABLE 5

| | | | Example | Example | Comparative | Comparative | Comparative | Comparative |
|---|---|---|---|---|---|---|---|---|
| | | Control | 9 | 10 | Example 4 | Example 5 | Example 6 | Example 7 |
| Ratio | Young barley leaf | | 100 | | | | | |
| (%) | powder | | | | | | | |
| of | Young barley leaf | | | 100 | | | | |
| each | juice powder | | | | | | | |
| test | (as juice ingredient) | | | | | | | |
| substance | Cellulose | | | | 100 | | | |
| | Vitamin D3 | | | | | 100 | | |
| | Spirulina | | | | | | 100 | |
| | β-glucan | | | | | | | 100 |
| | Total | 0 | 100 | 100 | 100 | 100 | 100 | 100 |

| | | | | | | | |
|---|---|---|---|---|---|---|---|
| NK cell cytotoxicity | 1.00 | 1.38 | 1.27 | 1.05 | 0.93 | 0.95 | 1.04 |
| (relative value) | | | | | | | |

### Test 4. Evaluation of Dendritic Cell Activity and Nk Cell Activity by Clinical Test

To confirm whether dendritic cells or NK cells are activated when a human ingests young barley leaves, a clinical test described below was conducted.

### <Test Method>

### 1) Test Food

For a test food, a powdered beverage was used in which young barley leaf powder (produced by the method described in Test 1) was mixed with maltose. For a control food, a powdered beverage was used that contained no young barley leaf powder, used a coloring agent and a flavoring agent in maltose and was adjusted to be visually indistinguishable from the test food. A daily ingestion amount for both the test food and the control food was designed to be 5.4 g. 2.7 g of each of the test food and the control food were placed in a plain aluminum pouch and distributed to a subject, and blinding of both the subject and an intervention implementer was ensured.

Calorie and nutrient composition values per daily ingestion (2 pouches) of the test food are shown in Table 6. The young barley leaf-derived dietary fiber contained in the test food was 0.7 g per daily ingestion (2 pouches).

TABLE 6

| | Control food | Test food |
|---|---|---|
| | (2 pouches) | (2 pouches) |
| Calories (kcal) | 21 | 20 |
| Protein (g) | 0.0 | 0.3 |
| Lipid (g) | 0.0 | 0.1 |
| Carbohydrate (g) | 5.3 | 4.7 |
| Sugar (g) | 5.3 | 4.0 |
| Dietary fiber (g) | 0.0 | 0.7 |
| Salt equivalent (g) | 0.000 | 0.002 |

Calories: energy conversion factors: protein (4), lipid (9), carbohydrate (4), dietary fiber (2)
Protein: nitrogen-protein conversion factor: 6.25
Dietary fiber: Prosky method

### 2) Subject

Paid volunteers were publicly recruited, and 56 individuals (19 males and 37 females) who met the following selection criteria and did not fall under the exclusion criteria were enrolled as subjects by a principal investigator. The subjects were provided with a sufficient explanation of the test content before the start of the test, and written consent was obtained.

Selection criteria: (1) healthy adult males and females aged 20 years or older and under 65 years at the time of obtaining consent; (2) those who have received sufficient explanation of the purpose and content of the research, have the capacity to consent, have volunteered to participate voluntarily after fully understanding, and have given written consent to participate in the research

Exclusion criteria: (1) those with serious diseases such as diabetes, kidney/liver diseases, or heart diseases, thyroid diseases, adrenal diseases, or other metabolic diseases, those under treatment, or those with a history of such diseases; (2) those with chronic diseases who regularly use medications; (3) those with digestive diseases that affect digestion and absorption, or those with a history of digestive system surgery; (4) those deemed unsuitable as research subjects based on blood tests conducted during screening; (5) those who have not experienced any subjective symptoms of upper respiratory tract infections in the winter for the past two years; (6) those with hay fever or allergic rhinitis who are taking medications; (7) those unable to stop taking supplements or health foods (including foods with nutrient function claims) that may affect the immune system; (8) those with excessive drinking habits exceeding about 60 g of pure alcohol equivalent per day; (9) smokers; (10) those with habits of intense exercise such as running or soccer; (11) those unable to abstain from alcohol from the day before screening and each examination; (12) those who reported having allergies to the ingredients of the research food; (13) those with a history or current condition of drug or alcohol dependence; (14) shift workers or night shift workers; (15) those planning to travel overseas; (16) those who are pregnant or breastfeeding, or who intend to become pregnant; (17) those who have used medications (including antiallergic drugs and antibiotics) that may affect immune function within two weeks of the screening; (18) those who have donated more than 200 mL of blood or blood components within two weeks or more than 400 mL of blood or blood components within three months of the screening; (19) those who have participated, or intend to participate, in other research within one month of obtaining consent; (20) others deemed unsuitable as research subjects by the principal investigator

### 3) Test Method

This test was conducted as a placebo-controlled, randomized, double-blind, parallel-group comparative study (assignment ratio: 1:1) with a total duration of 9 weeks consisting of a pre-observation period (1 week) and an ingestion period (8 weeks).

The principal investigator performed the enrollment according to the selection criteria and exclusion criteria, and a statistical analysis manager performed allocation using a block randomization method (with a block size of 4) with sex, age, and cDC activity markers as adjustment factors. A controller not directly involved in the test assigned the two allocated groups to a test food group and a control food group. Furthermore, the controller created and sealed a table (key code) describing the assignment result, and ensured blinding by keeping the key code sealed until it was disclosed after analysis subjects were determined.

During the test period, the subjects were instructed to ingest 2 pouches (5.4 g) of the test food (test food for the test food group and control food for the control food group) per day which was dissolved in an appropriate amount of water or hot water.

The subjects were provided with an explanation of things to note throughout the test period, such as that, during the test period, the same lifestyle as that before the start of the test should be maintained, use of supplements and health foods should be avoided, the frequency of ingesting foods containing lactic acid bacteria and bifidobacteria should be remained unchanged for those regularly ingesting these foods, excessive alcohol ingestion should be avoided, and participation in other tests should be avoided. In addition, the following were explained as things to note for each examination: alcohol ingestion should be avoided from the day before all examinations, sufficient sleep should be obtained the night before all examinations, consumption of anything other than water after 9 PM should be avoided on the day before all examinations, and intense exercise should be avoided from the day before until the completion of all examinations. The subjects were instructed to use pharmaceuticals only with the permission of the principal investigator or co-investigator, except in cases of emergency.

### 4) Examination Item

Measurements of cDC activity and activated NK cells were performed twice, at a pre-ingestion examination (hereinafter “pre-ingestion”) and at a post-ingestion examination (hereinafter “8 weeks post-ingestion”) after 8 weeks from the ingestion.

cDC activity was evaluated by measuring HLA-DR being a cDC activity marker in peripheral blood mononuclear cells (PBMCs). After PBMCs were isolated from blood, they were stained with CD4, CD141, CD11c, and HLA-DR antibodies, and measured using a flow cytometer. Among CD4-positive cells, those positive for both CD141 and CD11c were identified as cDCs, and a mean fluorescence intensity (MFI) of HLA-DR was calculated.

The activated NK cells were evaluated by measuring CD69-positive cells being an activation marker for NK cells. After PBMCs were isolated from blood, they were stained with CD3, CD45, CD56, and CD69R antibodies, and measured using a flow cytometer. Cells that were CD3-negative and positive for CD45, CD56, and CD69 were identified as activated NK cells, and the ratio of activated NK cells in lymphocytes was calculated.

### 5) Analysis Subject

The number of subjects enrolled in this test was 56 (19 males and 37 females), and the test was started with 56 subjects without any dropouts after randomization. During the test period, 4 subjects were determined by the principal investigator to meet discontinuation criteria and dropped out, resulting in 52 subjects completing the test. The reasons for meeting the discontinuation criteria and the number of subjects were as follows. 1 subject (test food group) became unwell due to reasons other than the test food and found it difficult to continue the test, 1 subject (control food group) was determined to have engaged in behavior that compromised their reliability as a subject, and 2 subjects (test food group) did not attend on the examination day.

After the test concluded, 8 subjects were found to meet rejection criteria, resulting in 44 subjects (15 males and 29 females) being included in the analysis. The reasons for meeting the rejection criteria and the number of subjects were as follows. (1) 7 subjects were deemed to violate the things to note during the test (changes in sleep habits: 1 subject in the test food group and 1 subject in the control food group; changes in lifestyle rhythm (work environment): 1 subject in the test food group and 4 subjects in the control food group); (2) 1 subject newly developed a disease (hay fever) during the test period (1 subject in the test food group).

### <Test Result>

An analysis result of the HLA-DR expression level being a cDC activity marker and the ratio of activated NK cells is shown in Table 7. With respect to both the HLA-DR expression level and the ratio of activated NK cells, the group that ingested the test food exhibited a significantly higher rate of change after 8 weeks from the ingestion compared to the control food group. It can be confirmed from this result that oral ingestion of young barley leaves by a human activates dendritic cells and NK cells.

TABLE 7

| Item | Group | Pre-ingestion | 8 weeks post-ingestion | Rate of change |
|---|---|---|---|---|
| HLA-DR MFI | Control food (n = 22) | 18842 | 27984 | 48.5% |
| | Test food (n = 22) | 19812 | 38551 | 94.6% |
| Ratio (%) of activated NK cells | Control food (n = 22) | 0.88 | 0.81 | −8.0% |
| | Test food (n = 22) | 1.00 | 1.23 | 23.0% |

The numerical values are average values

$$Rate of change ( \% ) = ( 8 weeks post - ingestion - pre - ingestion ) \div pre - ingestion \times 100$$

### Test 5. Production of Powdered Beverage

A powdered beverage of Example 11 consisting of the following ingredients was produced. Specifically, after each raw material was mixed, fluidized bed granulation was performed using a granulator, and a granular powdered beverage was produced. The produced powdered beverage was filled into aluminum pouches at 3 g per pouch. The obtained powdered beverage may be ingested by suspension in about 80 to 150 mL of water. By ingesting one package (0.3 g of young barley leaf powder, 0.135 g of young barley leaf-derived dietary fiber) of the powdered beverage of Example 11 per day, dendritic cells were activated, and effects of the present disclosure were achieved.

### Example 11

Young barley leaf powder (containing 45 mass % of dietary fiber): 10 mass %
Matcha: 4 mass %
Reduced maltose: 86 mass %

### INDUSTRIAL APPLICABILITY

The composition of the present disclosure exhibits an excellent dendritic cell activation effect by containing young barley leaves. Thus, according to the composition of the present disclosure, immune function can be maintained; furthermore, the effects such as antiviral action, anticancer action, promotion of healing of chronic wounds, skin whitening action, and anti-aging action on skin can be exerted. The composition of the present disclosure can be used as a food and beverage product aimed at dendritic cell activation or the like, and is therefore highly industrially useful.

1. A method for activating dendritic cells by causing a human to ingest young barley leaves.

2. The method according to claim 1, wherein immune function is maintained by activating dendritic cells.

3. A method for activating dendritic cells by causing a human to ingest young barley leaf-derived dietary fiber.

4. The method according to claim 3, wherein immune function is maintained by activating dendritic cells.

5. A method for activating natural killer cells, helper T cells, killer T cells, B cells, or macrophages by causing a human to ingest young barley leaves.
